(19) United States
(12) Patent Application Publication
(10) Pub. No.: US 2013/0297007 A1
(43) Pub. Date: Nov. 7, 2013

(54) APPARATUSES AND METHODS FOR GUIDING ENDOLUMINAL DEVICES THROUGH BODY LUMENS

(71) Applicant: Arun Kuchela, Portland, OR (US)

(72) Inventor: Arun Kuchela, Portland, OR (US)

(21) Appl. No.: 13/461,644

(22) Filed: May 1, 2012

(51) Int. Cl. A61F 2/84

(52) U.S. Cl. 623/1.23

(57) ABSTRACT

Apparatuses and methods for guiding endoluminal devices through body lumens. Several embodiments of the present technology, for example, are directed to flexible tubular sheaths or sleeves configured to provide a smooth, low friction pathway through which an endoluminal device can be delivered to a target site in a human patient. In one embodiment, for example, a flexible sheath or sleeve may comprise an elastomeric body and a plurality of structural elements carried by the elastomeric body. The structural elements may be oriented co-axially along the flexible sheath. The elastomeric body is configured to expand radially to accommodate passage of the endoluminal device through the flexible sheath or sleeve to the target site in the patient.

### TECHNICAL FIELD

The present technology relates generally to apparatuses and methods for guiding endoluminal devices through body lumens.

### BACKGROUND

Endoluminal devices, such as catheters, are widely used during surgical procedures for insertion into a variety of different body lumens. For example, such devices can be inserted or passed into vascular lumens (e.g., blood vessels or coronary arteries), non-vascular lumens (e.g., gastrointestinal lumens or the urethra, reproductive tracts), or other suitable body lumens.

During many procedures, however, the tortuosity of such lumens can hinder the delivery of the endoluminal device(s) to a target site. For example, vessel tortuosity, vessel calcification, vessel stiffness, and/or non-obstructing eccentric lesions can hinder delivery of the endoluminal devices. FIG. 1, for example, is a schematic, cross-sectional diagram of an endoluminal device 101 moving through a body lumen 104 using conventional techniques. In particular, the endoluminal device 101 moves out from a loader 102 and tracks over a guide wire 103 to advance through the body lumen 104 to a target site T. The guide wire 103 can have varying shaft thickness and rigidity. As shown in FIG. 1, a moving trajectory of the endoluminal device 101 is a combination of several shorter straight lines. This happens because when the guide wire 103 travels through a tortuous lumen 105 (e.g. a blood vessel), it will try to maintain a straight line and thus, by doing so, causes a wire-bias problem. In many instances, the wire bias problem may cause the endoluminal device 101 to hit an inner wall 107 of the body lumen 104 and/or encounter rough, eccentric wall lesions 106. The wire bias problem can prevent the endoluminal device 101 from a smooth passage to the target site T. Such impedance with delivery and retraction of the endoluminal device 101 can result in longer procedure times, and/or unintentional damage to non-target or target body tissue during the procedure.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent.

FIG. 1 is a partially schematic, cross-sectional view of a conventional endoluminal device moving through a body lumen.

FIG. 2 is a partially schematic, cross-sectional view of an endoluminal device and a flexible tubular sheath configured in accordance with several embodiments of the present technology.

FIG. 3A is a partially schematic, isometric view of the flexible tubular sheath of FIG. 2 external to the patient before deployment.

FIG. 3B is a top view of a proximal end of the flexible sheath of FIG. 3A.

FIG. 3C is a side view of the proximal end of the flexible sheath of FIG. 3A.

FIG. 4A is a partially schematic view of a distal end of the flexible sheath of FIG. 3A.

FIG. 4B is a partially schematic view of a distal end of a flexible tubular sheath configured in accordance with another embodiment of the present technology.

FIG. 5A is a partially schematic, cross-sectional end view of a flexible tubular sheath configured in accordance with an embodiment of the present technology before or after accommodating an endoluminal device.

FIG. 5B is a partially schematic, cross-sectional end view the flexible sheath of FIG. 5A when accommodating an endoluminal device.

FIG. 6A is a partially schematic, top view of a raw material used for manufacturing a flexible tubular sheath in accordance with an embodiment of the present technology.

FIG. 6B is a partially schematic, cross-sectional side view of the raw material shown in FIG. 6A.

FIG. 7A is a partially schematic, top view of a raw material used for manufacturing a flexible tubular sheath in accordance with another embodiment of the present technology.

FIGS. 7B-7E are partially schematic, cross-sectional side views of various embodiments of the raw material of FIG. 7A.

FIG. 8A is a partially schematic, cross-sectional end view of a flexible tubular sheath configured in accordance with still another embodiment of the present technology.

FIG. 8B is a partially schematic, cross-sectional end view of a flexible tubular sheath configured in accordance with yet another embodiment of the present technology.

FIG. 9 is a block diagram illustrating a method of applying and deploying a flexible tubular sheath in accordance with an embodiment of the present technology.

### DETAILED DESCRIPTION

The present technology is directed apparatuses and methods for guiding endoluminal devices through a body lumen. In particular, various embodiments of the present technology are directed to flexible tubular sheaths or sleeves configured to provide a smooth, low friction pathway through which an endoluminal device can be delivered to a target site in a patient. The flexible tubular sheath provides sufficient rigidity to bear a pushing force necessary to penetrate and move along inside a body lumen, while maintaining radially flexibility for accommodating endoluminal device during the delivery and retraction from the target site. The flexible tubular sheath is also configured to help mitigate the problems associated with delivery of endoluminal devices through tortuous body lumens. In addition, in several embodiments, the flexible tubular sheath is configured to provide radial flexibility to accommodate devices having various diameters and/or shapes. Accordingly, several embodiments of the present technology are expected to reduce invasive procedure times and unnecessary complications, inhibit or prevent damage to body lumens during delivery/retraction procedures, and are suitable for treatment of patients that have damaged and/or non-flexible vessels (e.g., elderly, diabetics, etc.).

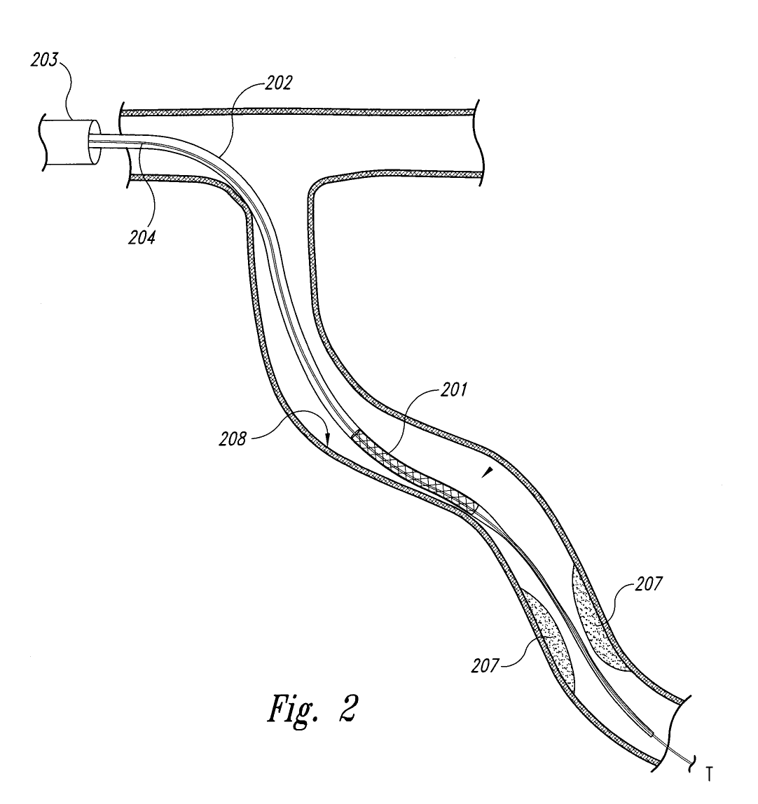

Specific details of several embodiments of the technology are described below with reference to FIGS. 2-9. Although many of the embodiments are described below with respect to devices, systems, and methods for guiding and delivering endoluminal devices through body lumens, other embodiments in addition to those described herein are within the scope of the technology. Additionally, several other embodiments of the technology can have different configurations, components, or procedures than those described herein. Other details describing well-known structures and devices often associated with endoluminal surgery or operation have not been set forth in the following disclosure to avoid unnecessarily obscuring the description of the various embodiments of the present technology. A person of ordinary skill in the art, therefore, will accordingly understand that the present technology may have other embodiments with additional elements, or the technology may have other embodiments without several of the features shown and described below with reference to FIGS. 2-9.

FIG. 2, for example, is a partially schematic, cross-sectional view of an endoluminal device 201 and a flexible sheath or sleeve 202 configured in accordance with several embodiments of the present technology. As shown in FIG. 2, the endoluminal device 201 is at least partially positioned inside the flexible sheath 202. The endoluminal device 201 may be, for example, a balloon catheter, a stent, or other such device. It will be appreciated that the flexible sheath 202 may be used with a wide variety of different devices suitable for insertion into body lumens of human patients. The flexible sheath 202 is configured to provide the endoluminal device 201 with a smooth, low-friction pathway toward a target site T in a human patient, and provide protection and guidance for the endoluminal device 201 while facilitating its movement toward the target site T. For purposes of this disclosure, the term “target site” is used to refer to any place within a body of a human patient that the endoluminal device may be delivered. In some embodiments (such as the embodiment illustrated in FIG. 2), the flexible sheath 202 may also slightly adjust the shape of the endoluminal device 201 to make it move smoothly along the pathway toward the target site T.

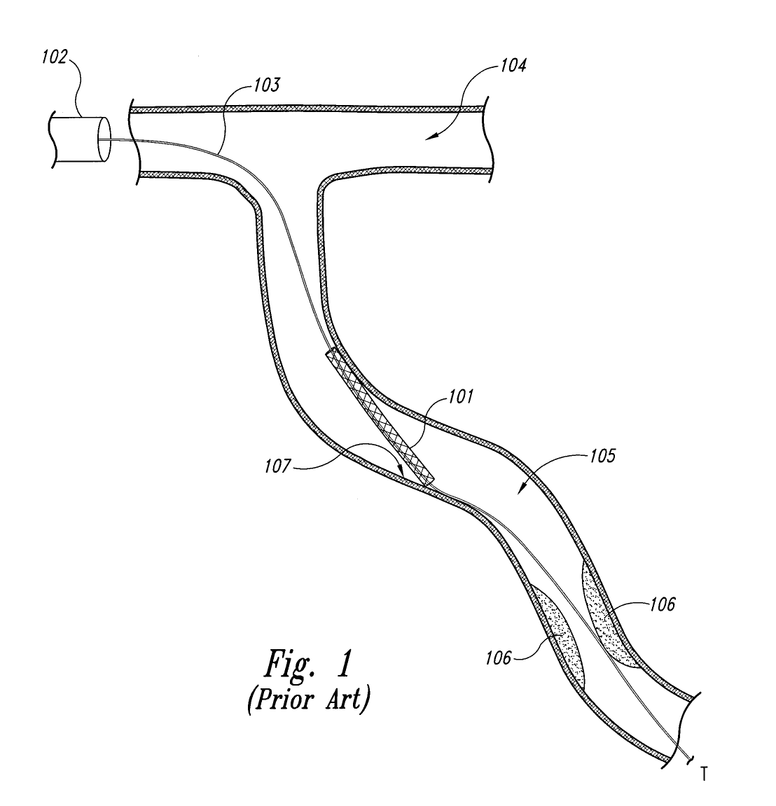

In operation, the endoluminal device 201 is configured to move out from a loader 203 and track over a guide wire 204 to advance through a body lumen 206 to the target site T. As shown in FIG. 2, the flexible sheath 202 is configured to pass through the tortuous lumen 206 relatively easily and smoothly, and without roughly scraping or damaging an inner wall 208 of the body lumen 206. The flexible sheath 202 is configured to substantially maintain a streamlined trajectory for the endoluminal device 201 through the body lumen 206 (rather than a combination of several straight lines associated with conventional devices as shown in FIG. 1). The flexible sheath 202 can also facilitate delivery of the endoluminal device 201 across a wall lesion 207 (e.g., a vessel lesion, a calcified non-obstructive lesion, etc.) without causing further damage to the lumen 206 and/or lesion 207. The flexible sheath 202 is accordingly expected to help prevent or inhibit the wire-bias problem associated with conventional techniques for delivering endoluminal devices as discussed above.

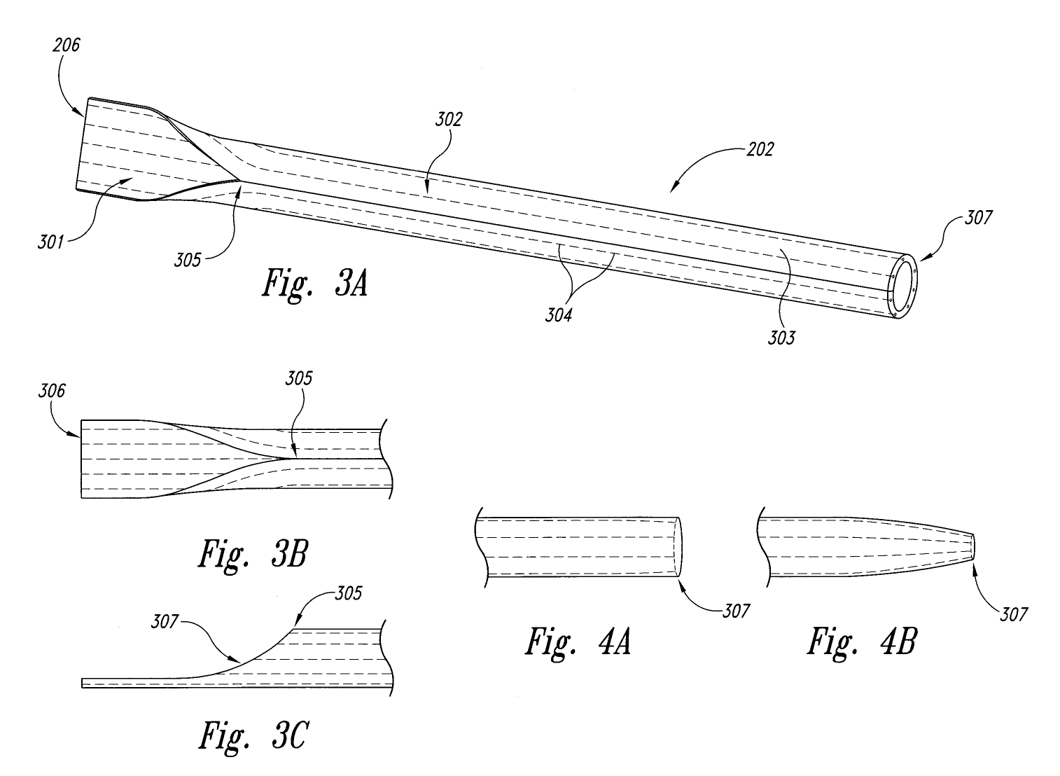

FIG. 3A is a partially schematic, isometric view of the flexible sheath or sleeve 202 of FIG. 2 before deployment into the patient. The flexible sheath 202 has an inner or internal surface 301 and an exterior or outside surface 302 opposite the inner surface 301. As described above with reference to FIG. 2, the endoluminal device 201 (FIG. 2) can be at least partially received within the flexible sheath 202 and in contact with the inner surface 301. In several embodiments, the inner surface 301 comprises a hydrophilic layer or coating to facilitate movement of the endoluminal device 201 inside the flexible sheath 202. The exterior surface 302 of the endoluminal device 202 can be configured to carry drugs and/or administer therapeutics to selected therapy sites within the patient by contacting with the inner wall 208 of the body lumen 206. In other embodiments, however, the exterior surface 302 may not include drugs or other agents.

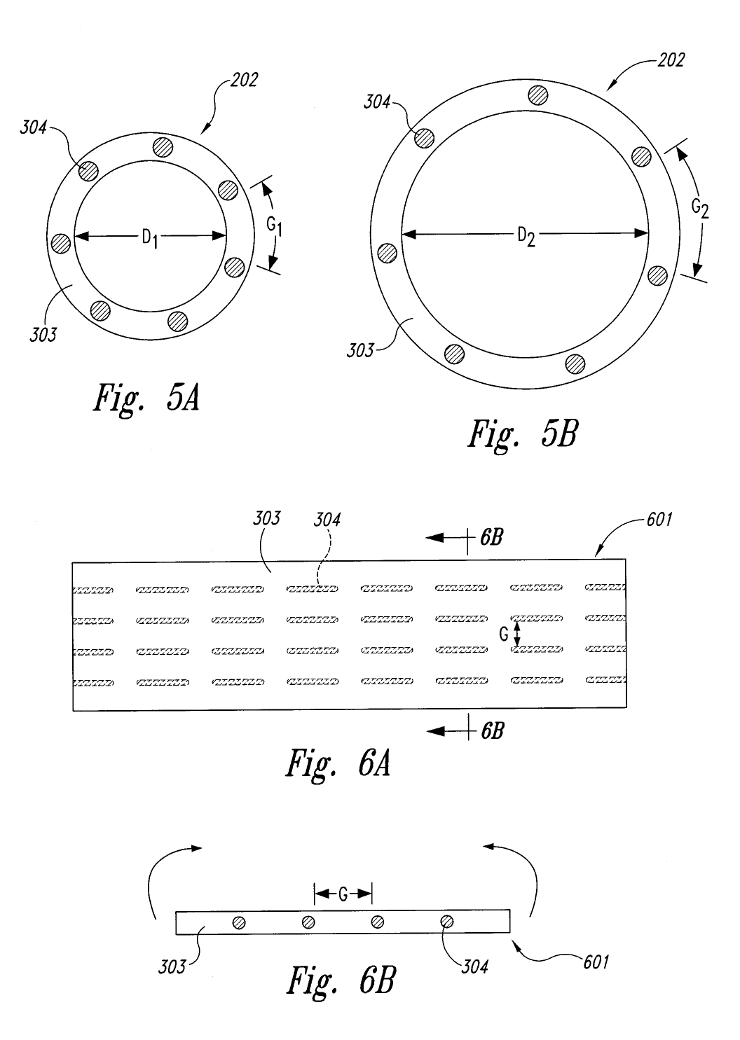

The flexible sheath 202 can include an elastomeric body 303 and a plurality of structural elements 304 disposed in and/or on the elastomeric body 303. For example, the structural elements 304 may be positioned within the elastomeric body 303 and oriented generally co-axially along the flexible sheath 202 and generally parallel with a longitudinal axis of the flexible sheath 202. In other embodiments, however, the structural elements 304 may have a different arrangement. Further, it will be appreciated that the arrangement and/or number of structural elements 304 in the embodiment of FIG. 3A is for illustrative purposes only, and the flexible sheath 202 may have a different number and/or arrangement of structure elements 304 in other embodiments. Further details regarding the arrangement of the structural elements 304 are described below with reference to FIGS. 5A-8B.

In several embodiments, the elastomeric body 303 may comprise an elastic material, such as polyurethane or other suitable materials. The structural element 304 may be comprised of metal or an alloy, such as tungsten, nickel titanium (i.e., nitinol), or other suitable materials. In still further embodiments, the elastomeric body 303 and/or structural elements 304 may be comprised of different materials.

In the embodiment described in FIG. 3A, the flexible sheath 202 is formed with a slit 305. The endoluminal device 201 can be positioned at least partially within and/or removed from the flexible sheath 202 via the slit 305. In several embodiments, the slit 305 extends generally co-axially along a length of the flexible sheath 202. In other embodiments, however, the size and/or shape of the slit 305 may vary (e.g., extend only along a portion of the length of the flexible sheath 202) depending on the arrangement of the endoluminal device 201 to be used with the flexible sheath 202. In still further embodiments, the slit 305 may have other arrangements and/or features, or the flexible sheath 202 may not include a slit 305.

The flexible sheath 202 includes a proximal end portion 306 and a distal end portion 307 opposite the proximal end portion 306. The proximal end portion 306 is at the “upstream” portion of the flexible sheath 202 and the distal end portion 307 is at the “downstream” portion of the flexible sheath 202. Referring to FIGS. 2 and 3A together, the endoluminal device 201 can enter the flexible sheath 202 at the proximal end portion 306 and move toward the distal end portion 307, via guidance of the flexible sheath 202, along the pathway to the target site T. When the endoluminal device 201 reaches the target site T, the flexible sheath 202 can be at least partially retracted and the endoluminal device 201 can move at least partially out from the distal end portion 307 of the flexible sheath 202. The extent to which the endoluminal device 201 moves out from the flexible sheath 202 can depend upon the configuration/type of the endoluminal device 201 in use. In several particular embodiments, for example, the endoluminal device 201 may be a balloon catheter and the device 201 can move partially out from the distal end portion 307 of the flexible sheath 202 to perform a predetermined operation. In other particular embodiments, the endoluminal device 201 can be a stent and can move entirely out from the flexible sheath 202 during operation and, in some instances, may be left at a desired site within the body lumen 206 to support the body lumen.

FIGS. 3B and 3C are top and side views, respectively, of the proximal end portion 306 of the flexible sheath 202. As shown in FIGS. 3B and 3C, the proximal end portion 306 may be tapered to facilitate placement and/or removal of the endoluminal device 201 (FIG. 2) relative to the flexible sheath 202. In several embodiments, for example, a tapered portion 307 can start from one end of the slit 305 and extend to the proximal end portion 306. In other embodiments, however, the arrangement of the tapered portion 307 can vary depending on the types of the endoluminal device 201 to be used with the flexible sheath 202. In still further embodiments, the flexible sheath 202 may not include the tapered portion 307.

FIGS. 4A and 4B are partially schematic views of the distal end portion 307 of the flexible sheath 202 in accordance with several embodiments of the present technology. In the embodiment shown in FIG. 4A, for example, the diameter of the flexible sheath 202 at the distal end portion 307 can be substantially the same as the average diameter of the other portions of the flexible sheath 202. In another embodiment shown in FIG. 4B, however, the distal end portion 307 may be tapered such that the diameter of the distal end portion 307 is smaller than the average diameter of the flexible sheath 202. In several embodiments, the smaller diameter at the distal end portion 307 (as shown in FIG. 4B) can help facilitate the flexible sheath 202 entering and moving along the body lumen 206. In some embodiments, however, the constant diameter arrangement (as shown in FIG. 4A) is expected to allow the endoluminal device 201 (FIG. 2) to easily exit the distal end portion 307 of the flexible sheath 202 during operation. Thus, it will be appreciated that arrangement of the distal end portion 307 of the flexible sheath 202 can vary depending on the types of endoluminal devices 201 (FIG. 2) to be used with the flexible sheath 202 and the size/configuration/tortuosity of the body lumen(s) into which the flexible sheath 202 will be delivered.

FIG. 5A is a partially schematic, cross-sectional end view of the flexible tubular sheath or sleeve 202 before or after accommodating a suitable endoluminal device (e.g., the endoluminal device 201 of FIG. 20, and FIG. 5B is a partially schematic, cross-sectional end view the flexible sheath of FIG. 5A in an expanded arrangement when accommodating the endoluminal device. Referring to FIGS. 5A and 5B together, the flexible sheath 202 includes the elastomeric body 303 and the structural elements 304 on a cross-sectional plane. The flexibility of the elastomeric body 303 allows the flexible sheath 202 to expand axially to accommodate the endoluminal device 201. At the same time, the structural elements 304 provide the flexible sheath 202 with the necessary strength/rigidity to move through the body lumen 206 and to guide the endoluminal device 201 (FIG. 2).

The flexible sheath 202 can have a first inner diameter D₁ (in FIG. 5A) before/after accommodating the endoluminal device. Gaps G₁ (FIG. 5A) and G₂ (FIG. 5B) can be defined as the distance between each two structural elements 304 on one cross-sectional plane of the flexible sheath 202. Referring to FIG. 5B, to accommodate the endoluminal device (not shown) the elastomeric body 303 expands such that it has a second inner diameter D₂ greater than the first inner diameter D₁. The gaps enlarge from G₁ to G₂ while the inner diameter increases from D₁ from D₂. After the endoluminal device passes through the cross section, the gaps and inner diameter can return to their original state (back to G₁ and D₁, respectively).

As shown in FIGS. 5A and 5B, the gaps G₁ and G₂ between the individual structural elements 304 remain substantially the same among the structural elements 304 on each cross-sectional plane. However, in other embodiments, the gaps G₁ and G₂ between the individual structural elements 304 may vary depending upon the different arrangements/configurations of the structural elements 304. Different arrangements of the structural elements 304 are described in greater detail below with reference to FIGS. 7A-8B.

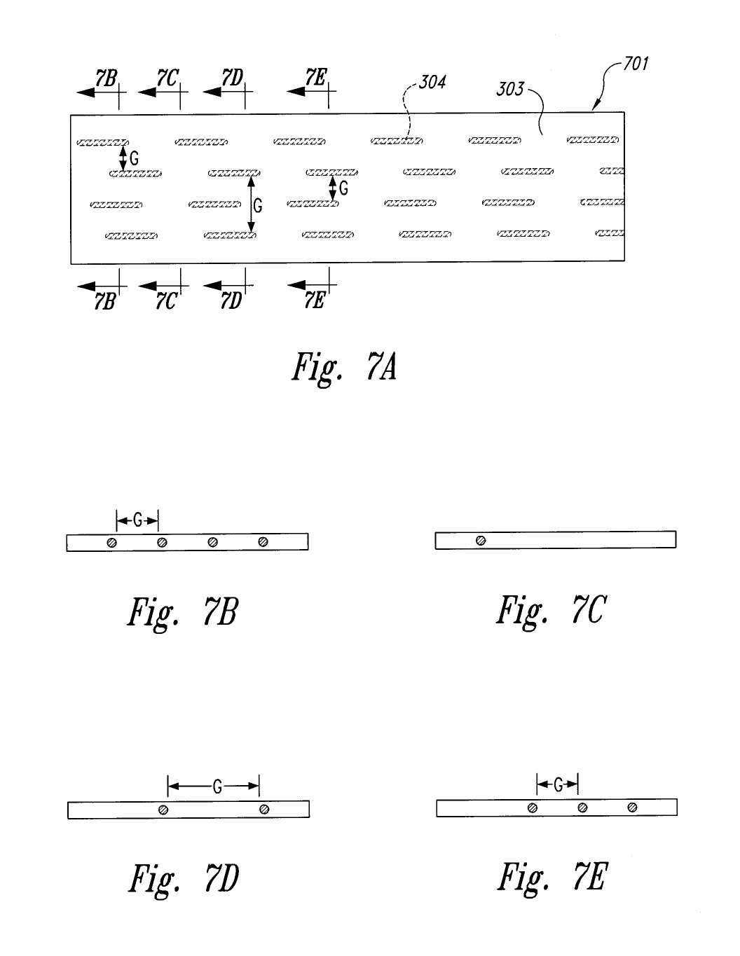

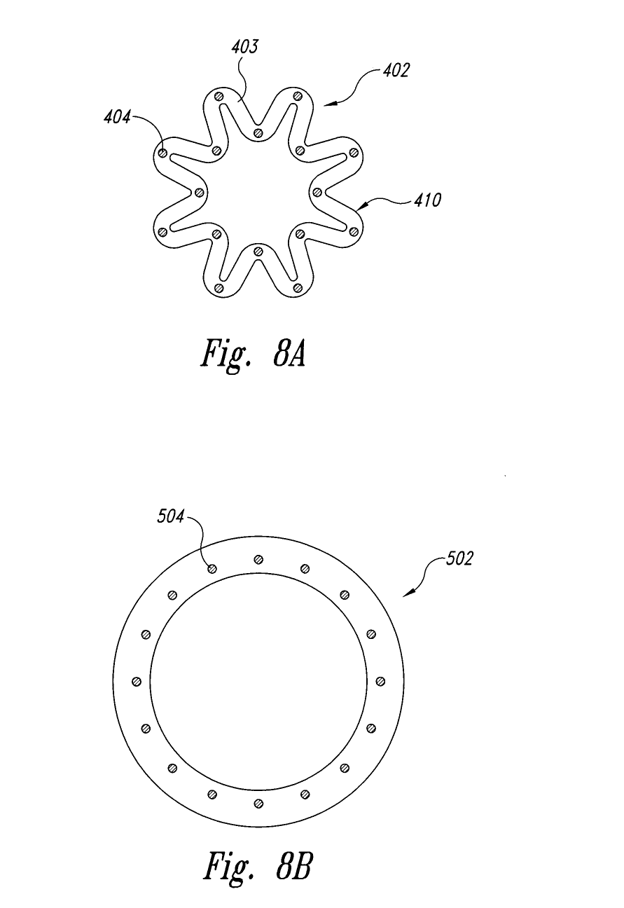

FIG. 6A is a top view of a raw material 601 used for manufacturing the flexible sheath 202 (FIGS. 2 and 3A) in accordance with several embodiments of the present technology. The raw material 601 can include, for example, the elastomeric body 303 and a plurality of structural elements 304 positioned on and/or in the elastomeric body 303. In this embodiment, the gaps G between each two structural elements 304 are substantially the same. FIG. 6B is a cross-sectional view of the raw material 601 shown in FIG. 6A. The raw material 601 can be rolled up (following the directions shown by the arrow signs in FIG. 6B) and then fixed in place to form the flexible sheath 202. In some embodiments, for example, the raw material 601 can be fixed by adjusting suitable working temperatures or by other suitable methods known to the persons having ordinary skill in the art. In several embodiments, when fixing the raw material 601, the slit 305 (FIG. 3A) can be formed. As described above, dimensions of the slit 305 (FIG. 3a) can vary depending on the size and scale of the endoluminal device(s) to be accommodated by the flexible sheath 202 (FIG. 3A).

FIG. 7A is a top view of a raw material 701 used for manufacturing the flexible sheath 202 in accordance with another embodiment of the present technology. The raw material 701 can include the elastomeric body 303 and the structural elements 304 placed on and/or in the elastomeric body 303. This embodiment differs from the embodiment described above with reference to FIGS. 6A and 6B in that the gaps G between structural elements 304 can vary depending upon the location of the structural elements. For example, FIGS. 7B, 7C, 7D and 7E are cross-sectional views of the raw material 701 shown in FIG. 7A. As shown in FIGS. 7B, 7C, 7D and 7E, different cross-sections of the raw material 701 can have different numbers of structural elements 304, and different gaps G between individual structural elements 304. Different arrangements of the structural elements 304 are expected to provide different strength and/or flexibility for the resulting flexible sheath fabricated from the raw material 701. For example, the strength/rigidity of the cross-section in FIG. 7B is greater than the strength of the cross-section of FIG. 7C because there are more structural elements 304 in the cross-section in FIG. 7B. On the contrary, the cross-section in FIG. 7C has great flexibility than the cross-section in FIG. 7B, because there is more elastomeric body 303 (and less structural elements 304) in the cross-section of FIG. 7C. Therefore, the flexible sheath 202 can have various levels of strength and/or flexibility on different cross-sectional planes along a length of the flexible sheath 202, and thus can accommodate various types of endoluminal devices and be used in a wide variety of different body lumens.

FIG. 8A is a partially schematic, cross-sectional end view of a flexible tubular sheath or sleeve 402 configured in accordance with still another embodiment of the present technology. The flexible sheath 402 can include several features generally similar to the flexible sheath 202 described above. For example, the flexible sheath 402 includes an elastomeric body 403 and a plurality of structural elements 404. The cross-sectional shape of the flexible sheath 402, however, can be different than the flexible sheath 202 described above. For example, the flexible sheath 402 can have an “octagonal-star” cross-section, i.e., it has eight corners 410 on a cross-sectional plane. It will be appreciated that the arrangement of the flexible sheath 402 shown in FIG. 8A is only an exemplary illustration, and the number of corners 410 can vary in other embodiments according to the need for accommodating different types of endoluminal devices and/or body lumens. The non-circular design of the flexible sheath 402 is expected to provide the flexible sheath 402 with an enhanced range of flexibility, and can allow the flexible sheath 402 to be used with endoluminal devices having a variety of different arrangements and configurations. In still other embodiments, the flexible sheath or sleeve 402 may have a variety of other non-circular cross-sectional shapes (e.g., triangular, rectangular, etc.) in addition to or in lieu of the octagonal-star shape described above.

FIG. 8B is a partially schematic, cross-sectional end view of a flexible tubular sheath 502 configured in accordance with yet another embodiment of the present technology. In the embodiment shown in FIG. 8B, the flexible sheath 502 includes a plurality of structural elements 504 having a different arrangement from the embodiments described above. The numbers and/or shapes of the structural elements 504 can be varied to provide the flexible sheath 502 a wide range of strength and/or flexibility. In the illustrated embodiment, for example, the structural elements 504 have a smaller cross-sectional dimension than the structural elements 304 described above with reference to FIGS. 5A and 5B. In other embodiments, however, the cross-sectional size of the structural elements 504 can be varied. The cross-sectional shape of the structural elements 504 can also be varied. For example, the structural elements 504 can have a variety of different cross-sectional shapes (e.g., circular, triangular, rectangular, etc.). In addition, the shapes and/or sizes of the structural elements 504 need not to be the same on one cross-sectional plane of the flexible sheath 502. For example, the shapes of the structural elements 504 on one cross-sectional plane of the flexible sheath 502 can include circle, triangles, rectangles, and/or other shapes to impart a desired strength and flexibility to the flexible sheath 504 based, at least in part, on the endoluminal device(s) to be used and the configuration of the target body lumen(s).

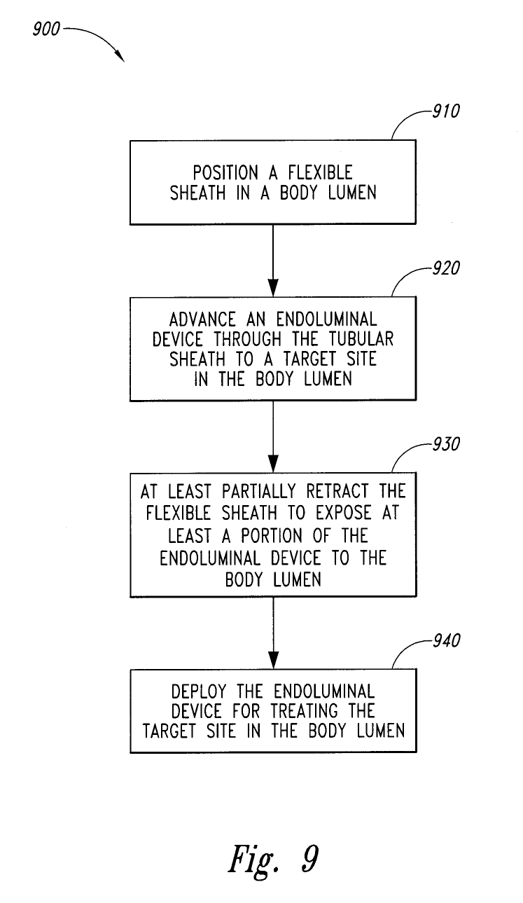

FIG. 9 is a block diagram illustrating a method 900 of applying and deploying a flexible tubular sheath (e.g., the flexible sheaths 202/402/502 or other suitable flexible sheaths) in accordance with an embodiment of the present technology. The method 900 will be described in accordance with the flexible sheath or sleeve 202 and endoluminal device 201 of FIG. 2 above. It will be appreciated, however, that the method 900 may be utilized with any suitable flexible tubular sheath or sleeve configured in accordance with the disclosed technology (e.g., the flexible sheaths 402/502 or other suitable flexible sheaths) and any type of suitable endoluminal devices.

Referring to FIGS. 2 and 9 together, the method 900 starts at block 910 by positioning the flexible sheath or sleeve 202 in a body lumen 206. The distal end portion of the flexible sheath 202 enters the body lumen 206 from the loader 203. In several embodiments, the endoluminal device 201 can be positioned in the flexible sheath 202 before the step described in block 910. In other embodiments, however, the endoluminal device 201 can be positioned in the flexible sheath 202 after the step described in block 910. In several embodiments, the method 900 can further delivering the endoluminal device 201 to the flexible sheath 202 through the slit 305.

The method 900 continues at block 920 by advancing the endoluminal device 201 to the target site T in the body lumen 206 through the flexible sheath 202. As described above, the flexible sheath 202 provides the endoluminal device 201 with protection and guidance to facilitate its movement to the target site T. The method 900 continues at block 930 with at least partially retracting the flexible sheath 202 to expose the endoluminal device 201 within the body lumen 206. At block 940, the method 900 further includes deploying the endoluminal device 201 for therapy/treatment at the target site T in the body lumen 206. In some embodiments, the method 900 can optionally include retracting the endoluminal device 201 into and through the flexible sheath 202 upon completion of the therapy/treatment. In other embodiments, however, the method 900 may include leaving the endoluminal device 201 at the target site T.

From the foregoing, it will be appreciated that specific embodiments of the technology have been described herein for purposes of illustration, but that various modifications may be made without deviating from the disclosure. For example, as described above, flexible sheaths configured in accordance with the present technology can have a variety of different shapes, sizes, and arrangements based upon the types of endoluminal devices that may be used with the sheaths and/or the arrangement of the body lumens into which the sheaths will be delivered. It will be further appreciated that the shapes of the flexible sheaths and structural elements described above are not limited by the embodiments disclosed herein, and a variety of different shapes/configurations may be used. Certain aspects of the new technology described in the context of particular embodiments may be combined or eliminated in other embodiments. In addition, while advantages associated with certain embodiments of the new technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. An apparatus for guiding an endoluminal device through a body lumen of a human patient, the apparatus comprising:
a flexible sheath configured for placement within the body lumen of the patient, wherein the flexible sheath comprises an inner surface configured to receive the endoluminal device and an exterior surface opposite the inner surface, the flexible sheath including—
an elastomeric body; and
a plurality of structural elements carried by the elastomeric body,
wherein the elastomeric body is configured to expand radially to accommodate the endoluminal device as it passes through the flexible sheath to a target site in the patient.

2. The apparatus of claim 1 wherein the inner surface has a hydrophilic coating.

3. The apparatus of claim 1 wherein the exterior surface comprises a drug or agent, and wherein the flexible sheath is further configured to deliver a drug or agent to a vessel wall of the body lumen.

4. The apparatus of claim 1 wherein the flexible sheath comprises a slit for facilitating positioning of the endoluminal device at least partially inside the flexible sheath and removal of the endoluminal device from the flexible sheath.

5. The apparatus of claim 1 wherein the flexible sheath includes a distal end portion having a diameter less than an average diameter of the flexible sheath.

6. The apparatus of claim 1 wherein the flexible sheath includes a proximal end portion with a tapered portion to facilitate placement and/or removal of the endoluminal device from the flexible sheath.

7. The apparatus of claim 1 wherein the sheath has a sheath length, and wherein the structural elements have an element length less than the sheath length, and further wherein the individual structural elements are spaced apart from each other by gaps.

8. The apparatus of claim 1 wherein the individual structural elements have a generally circular cross-sectional shape.

9. The apparatus of claim 1 wherein the flexible sheath is expandable from a first diameter to a second diameter greater than the first diameter as the endoluminal device passes along the inner surface of the flexible sheath to the target site while the flexible sheath is within the body lumen of the patient, and further wherein the flexible sheath is configured to return to the first diameter after passage of the endoluminal device.

10. The apparatus of claim 1 wherein:
a first portion of the flexible sheath has a first rigidity based, at least in part, on the arrangement of the structural elements at the first portion of the flexible sheath; and
a second portion of the flexible sheath has a second rigidity different than the first rigidity based, at least in part, on the arrangement of the structural elements at the second portion of the flexible sheath.

11. A method, comprising:
positioning an endoluminal device inside a flexible sleeve positioned within a body lumen, wherein the flexible sleeve includes an elastomeric body and a plurality of structural elements carried by the elastomeric body and oriented approximately parallel with a longitudinal axis of the flexible sleeve, and wherein the elastomeric body is configured to radially expand to accommodate the endoluminal device;
advancing the flexible sleeve and the endoluminal device to a desired location in the body lumen; and
at least partially retracting the flexible sleeve to expose the endoluminal device to the body lumen.

12. The method of claim 11, further comprising deploying the endoluminal device from the flexible sleeve and treating a target site in the body lumen.

13. The method of claim 11, further comprising retracting the flexible sleeve and the endoluminal device through the body lumen.

14. The method of claim 11 wherein an inner surface of the flexible sleeve has a hydrophilic coating.

15. The method of claim 11 wherein the flexible sleeve comprises a drug or agent, and wherein the method further comprises delivering the drug or agent to a desired site along the body lumen while the sleeve is within the body lumen.

16. The method of claim 11 wherein the flexible sleeve comprises a slit, and wherein positioning the endoluminal device inside a flexible sleeve comprises passing at least a portion of the endoluminal device through the slit.

17. The method of claim 11 wherein the flexible sleeve has a distal end portion with a diameter less than an average diameter of the flexible sleeve, and wherein at least partially retracting the flexible sleeve to expose the endoluminal device to the body lumen comprises passing the endoluminal device through the reduced diameter distal end portion.

18. The method of claim 11 wherein the flexible sleeve has a tapered proximal end portion, and wherein positioning the endoluminal device inside the flexible sleeve comprises passing the endoluminal device through the tapered portion of the sleeve.

19. The method of claim 11, further comprising completely removing the flexible sleeve from the patient after treatment.

20. The method of claim 11 wherein advancing the flexible sleeve and the endoluminal device to a desired location in the body lumen comprises moving the endoluminal device along a streamlined trajectory through the body lumen.
